(19) United States
(12) Patent Application Publication
(10) Pub. No.: US 2008/0108612 A1
(43) Pub. Date: May 8, 2008

(54) Use of Purine Derivatives as HSP90 Protein Inhibitors

(75) Inventors: Chantal CARREZ, Thiais (FR); Florence FASSY, Paris (FR); Patrick MAILLIET, Fontenay Sous Bois (FR)

Correspondence Address: ANDREA Q. RYAN;SANOFI-AVENTIS U.S. LLC, 1041 ROUTE 202-206, MAIL CODE: D303A, BRIDGEWATER, NJ, 08807, US

(73) Assignee: AVENTIS PHARMA S.A., Antony (FR)

(21) Appl. No.: 11/773,577

(22) Filed: Jul. 5, 2007

Related U.S. Application Data
(63) Continuation of application No. PCT/FR06/00066, filed on Jan. 11, 2006.

(30) Foreign Application Priority Data
Jan. 13, 2005 (FR) 0500350

(51) Int. Cl. A61K 31/52; A61K 31/5377; A61P 35/00

(52) U.S. Cl. 514/234.2; 514/252.16; 514/263.3; 514/263.4

(57) ABSTRACT

This invention relates to methods of inhibiting the Hsp90 chaperone protein, and methods of treatment comprising administration of compounds of formula (IA) (IB) and (II)

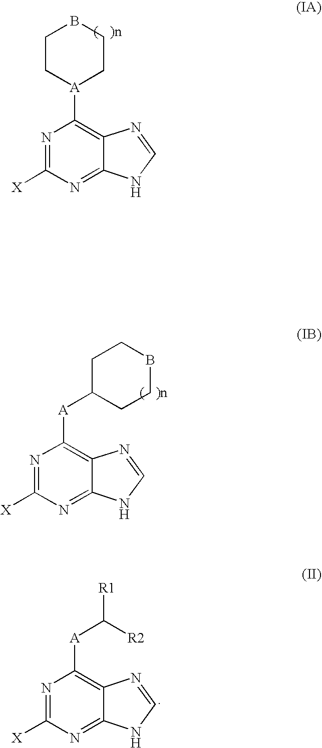

The invention relates to the use of purine derivatives as inhibitors of the activity of the Hsp90 chaperone protein, and more particularly their use as inhibitors of the ATPase-type catalytic activity of the Hsp90 chaperone protein.

The present invention especially relates to the use of purine derivatives as an anticancer agent and one subject of the present invention is also the use of purine derivatives to obtain a medication intended for the treatment of cancer.

Another subject of the invention is the use of purine derivatives and their pharmaceutically acceptable salts for the preparation of pharmaceutical compositions intended to treat diseases in which an abnormal activity of the Hsp90 protein is involved.

The purine derivatives in question in the present invention correspond to the following general formulae (IA), (IB) or (II):

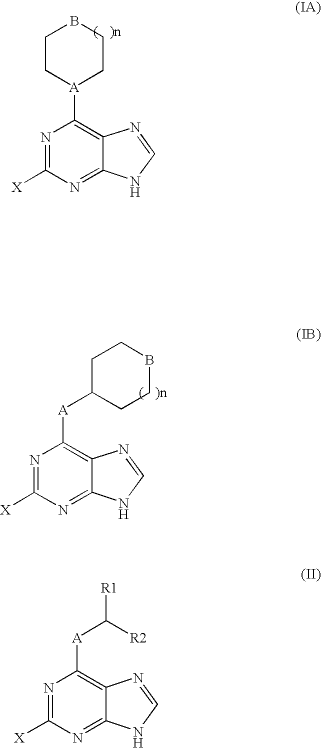

Patent Application EP 300 726 claims piperazinyl derivatives of purines as hypoglycemic agents.

Patent Application WO 04/035740 claims amino-morpholinopurine derivatives useful for treating pathologies linked to interleukin-12 (IL-12) overproduction.

Patent Application WO 02/051843 claims a preparation method for purine derivatives and also the use of these derivatives as antifungal agents.

The present invention relates to the use of products of general formula (IA), (IB), or (II) below:

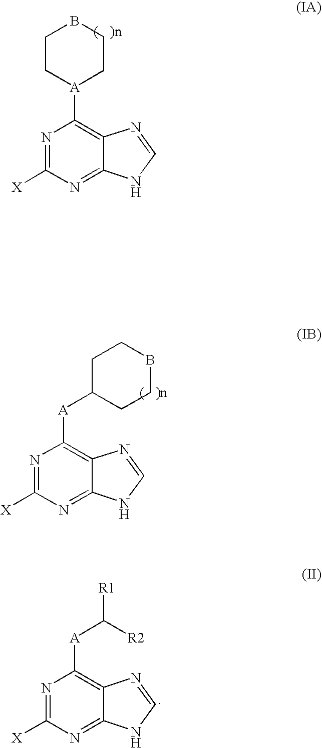

in which:
1) when the general formula is (IA) or (IB), X represents a hydrogen atom, a methyl or trifluoromethyl radical;
2) when the general formula is (II), X represents a hydrogen or halogen atom, a methyl or trifluoromethyl radical;
3) when the general formula is (IA), A represents N or CH;
4) when the general formula is (IB), A represents O, S, NH, CH₂ or CHR;
5) when the general formula is (II), A represents O, S, NH, NR1, CH₂ or CHR1;
6) B represents O, S, NR′, CH₂ or CHR′;
7) R represents a hydrogen atom, or a C₁-C₃ alkyl radical;
8) R′ represents a hydrogen atom, or a C₁-C₇ alkyl radical, or a C₂-C₇ alkenyl or alkynyl radical, or a (CH₂)ₙ-aryl or heteroaryl radical, or a C(Z)-aryl or heteroaryl radical; the aryl or heteroaryl rings, being monocyclic or bicyclic with 5 to 10 ring members, may contain from 0 to 3 identical or different heteroatoms chosen from O, S or N, and may optionally be substituted by one or more halogen atoms or by one or more radicals chosen from the group composed of alkyl, OH, Oalkyl, SH, Salkyl, NH₂, NHalkyl, N(alkyl)₂, CF₃, CN, NO₂, COOH, C(O)Oalkyl, CONH₂, C(O)NHalkyl, C(O)N(alkyl)₂, S(O)alkyl, S(O)₂alkyl, SONH₂, S(O)₂NH₂, S(O)₂NHalkyl, S(O)₂N(alkyl)₂, —C(O)NH₂, P(O)(OH)₂, P(O)(alkyl)OH, P(O)(Oalkyl)₂, P(O)(alkyl)Oalkyl, NH—C(O)—NH₂, NH(CO)NHalkyl, NH(CO)N(alkyl)₂, O—C(O)NHalkyl, O—C(O)N(alkyl)₂, of which the alkyl parts may be C₁-C₃;
9) n=0, 1 or 2;
10) Z represents an oxygen or sulfur atom or an NR′ radical with R′ as defined previously;
11) R1 represents a hydrogen atom or a C₁-C₃ alkyl radical; and
12) R2 represents a C₁-C₃ alkyl radical or a CHR1-aryl or heteroaryl ring; the aryl or heteroaryl ring, being monocyclic or bicyclic with 5 to 10 ring members, may contain from 0 to 3 identical or different heteroatoms chosen from O, S or N; the alkyl radical or the aryl or heteroaryl ring may optionally be substituted by one or more halogen atoms or by one or more radicals chosen from alkyl, OH, Oalkyl, SH, Salkyl, NH₂, NHalkyl, N(alkyl)₂, CF₃, CN, NO₂, COOH, C(O)Oalkyl, CONH₂, C(O)NHalkyl, C(O)N(alkyl)₂, S(O)alkyl, S(O)₂alkyl, SONH₂, S(O)₂NH₂, S(O)₂NHalkyl, S(O)₂N(alkyl)₂, —C(O)NH₂, P(O)(OH)₂, P(O)(alkyl)OH, P(O)(Oalkyl)₂, P(O)(alkyl)Oalkyl, NH—C(O)—NH₂, NH(CO)NHalkyl, NH(CO)N(alkyl)₂, O—C(O)NHalkyl, O—C(O)N(alkyl)₂, of which the alkyl parts may be C₁-C₃,
   in racemic form, enriched in one enantiomer, enriched in one diastereoisomer, its tautomers, its prodrugs and its pharmaceutically acceptable salts, for the preparation of medications useful for treating diseases in which an abnormal activity of the Hsp90 protein is involved.

The present invention also relates to the use of the products of general formulae (IA), (IB) or (II) as defined above for the manufacture of a medication useful for treating a pathological condition, preferably cancer.

Among the compounds that are useful according to the invention and are particularly preferred, mention may be made of the following compounds:

Products of general formula (IA) or (IB) for which X represents a hydrogen atom are preferred.

As examples of the halogen atom which X may represent, mention may be made of chlorine (Cl), fluorine, bromine or iodine.

Products of general formula (II) for which X═Cl are preferred.

As examples of aryl or heteroaryl rings that are monocyclic or bicyclic with 5 to 10 ring members and which may contain from 0 to 3 identical or different heteroatoms chosen from O, S or N, which may optionally be substituted, mention may be made of phenyl, pyridyl, pyrimidine, triazine, pyrrolyl, imidazolyl, thiazolyl, pyrrazolyl, furyl, thienyl, indolyl, indazolyl, azaindolyl, isobenzofuranyl, isobenzothienyl, benzoxazolyl, benzothiazolyl, quinolelyl, arylvinylene, arylamido, arylcarboxamide, aralkylamine, quinoleyl, isoquinoleyl, cinnolyl, quinazolyl, naphtyridyl, triazolyl or tetrazolyl groups.

From the compounds of formula (IA) it is preferred to choose those for which A=N.

Products of general formula (IA) for which A=N are preferred.

Products of general formula (IA) for which A=N, B═NR′ are preferred.

Products of general formula (IA) for which A=N, B═NR′ and n=1 are preferred.

When B is NR′ or CHR′, a preferred substituent R′ could be chosen from phenyl, phenyl substituted by at least one radical chosen from a halogen atom, Oalkyl, —C(O)NH₂, or phenylmethyl, or phenylamino, or pyridyl, or pyrimidinyl or quinolinyl.

More preferably, from the products of general formula (IA) those for which X═H, A=N and n=1 are preferred.

It is also preferred to choose from the products of formula (IB) those for which A=NH.

Products of general formula (IB) for which A=NH, B═CH₂ are preferred.

Products of general formula (IB) for which A=NH, n=0 are preferred.

Products of general formula (IB) for which X═H, A=NH are preferred.

It is also preferred to choose from the products of formula (II) those for which A=NH and more particularly those for which X═Cl and A=NH.

It is also preferred to choose from the products of formula (II) those for which A=CH₂.

Products of general formula (II) for which A=NH, R1=H are preferred.

Among the compounds of formula (IA), (IB), or (II) used according to the invention, mention may be made of the following compounds:
6-(phenylmethyl)amino-1H-purine monohydrochloride
2-chloro-6-phenylmethyloxy-1H-purine
2-chloro-6-(1(R,S)-phenylethyl)amino-1H-purine
2-chloro-6-[2-(morpholin-4-yl)ethylamino]-1H-purine
6-(thiophen-2-yl)methylamino-1H-purine
2-chloro-6-[2-(phenylmethylamino)ethylamino]-1H-purine
6-{2-[3-(3,5-dimethylphenyl)oxypropyl]amino}-1H-purine
6-[4-(ethyloxycarbonyl)methylpiperidin-1-yl]1H-purine
6-(piperidin-1-yl)-1H-purine
6-[4-(pyridin-2-yl)piperazinyl]-1-H-purine monohydrochloride.

Generally, products of general formula (IA) or (IB) according to the invention in which A is a nitrogen atom may be prepared by action of a primary or secondary amine on a 2,6-dihalopurine (or a 6-halopurine), according to the scheme 1, in particular by using the method described in J. Amer. Chem. Soc. (1959), 81, 3789-92.

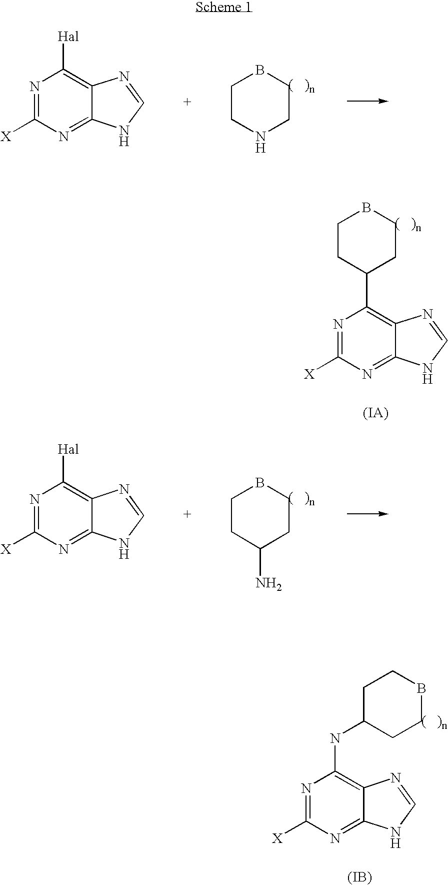

The compounds of general formula (IA) or (IB) in which A is a CH radical may be prepared by coupling, in the presence of a catalyst such as palladium tetrakis-(triphenylphosphine), an organometallic cycloalkane or heterocycloalkane derivative (with B═CH₂, CHR, O, S, NH or NR) to a 2,6-dihalopurine (or a 6-halopurine), of which the nitrogen atom in position 7 will have been previously protected, according the scheme 2, in particular by using an organozinc compound according to the method described in Nucleoside, Nucleotide & Nucleic acids 2000, 1123.

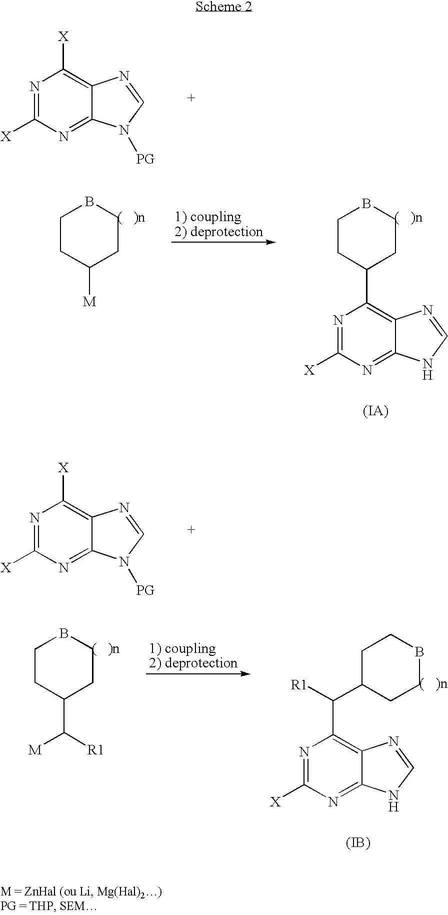

The compounds of general formula (IB) in which A is an oxygen or sulfur atom may be prepared by action of an alkali metal or alkaline-earth metal alcoholate or thioalcoholate on a 2,6-dihalopurine (or a 6-halopurine) according to the Scheme 3, in particular by using the method described in Tetrahedron Lett., 2001, 8161.

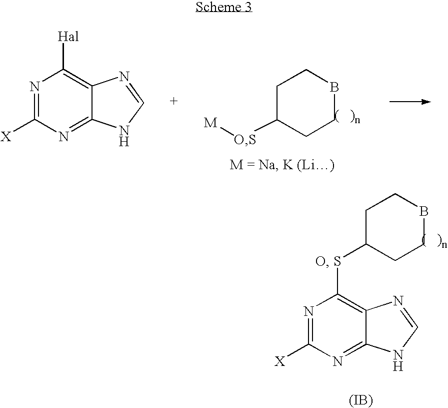

The products of general formula (II) may be obtained according to the methods described in the literature, such as, for example WO 2001/049688, WO 98/05335, JP 04005290, U.S. Pat. No. 6,096,724, U.S. Pat. No. 5,929,046 or Tetrahedron Lett., 2001, 8161.

The examples below illustrate, nonlimitingly, the products of the invention.

### EXAMPLE 1

### 6-(phenylmethyl)amino-1H-purinemonohydrochloride

In a 50 ml round-bottomed flask, 500 mg of 2,6-dichloro-1H-purine were dissolved in 10 ml of butanol and 1 ml of propan-2-ol, then 620 μl of 4-(phenylmethyl)piperazine were added and the mixture was heated to 75° C. After heating for about 3 h, a white precipitate began to appear. After heating for 4 h, the reaction was complete (TLC on 60F254 silica plate—dichloromethane/methanol eluent, 90/10 by volume). After cooling to 10° C., the precipitate formed was filtered off then washed successively with 0.5 ml of butanol, 2 times 1 ml of methanol and 2 times 1 ml of diethyl oxide. Thus 720 mg of 2-chloro-6-[4-(phenylmethyl)-piperazin-1-yl]-1H-purine monohydrochloride was obtained, in the form of a yellow powder, the characteristics of which were the following:

Melting point (Kofler)=258-60° C.

Mass spectrum (EI): m/z=294 (M+).

### EXAMPLE 10

6-[4-(pyridin-2-yl)piperazinyl]-1-H-purine monohydrochloride, was obtained by proceeding according to Example 1 but by replacing the 4-(phenylmethyl)piperazine with 4-(pyridin-2-yl)piperazine.

### EXAMPLE 2

### 2-chloro-6-phenylmethyloxy-1H-purine

In a 50 ml round-bottomed flask, 500 mg of 2,6-dichloro-1H-purine were dissolved in 10 ml of tetrahydrofuran, added next was a solution of sodium phenylmethanolate, prepared at the time of use from 314.7 mg of phenylmethanol and 116 g of sodium hydride (as a 60% mixture in oil) in 10 ml of tetrahydrofuran and the mixture was refluxed for 20 hours. After cooling, the reaction medium was concentrated under reduced pressure, then extracted 3 times with 20 ml of dichloromethane. The combined organic phases were washed with water, dried over magnesium sulfate and concentrated to dryness under reduced pressure. After purifying by flash chromatography over silica gel while eluting with dichloromethane, 195 mg of 2-chloro-6-phenylmethyloxy-1H-purine was obtained, in the form of a white powder, the characteristics of which were the following:

Melting point (Kofler) 126-28° C.

Mass spectrum (EI): m/z=273 (M+).

2-chloro-6-phenylmethyloxy-1H-purine is cited, for biological activities different than those claimed in the present invention, in Nucleotides & Nucleosides 1999, 18(4-5), 873-74 without references to its preparation nor to its physicochemical characteristics.

### EXAMPLE 3

### 2-chloro-6-(1(R,S)-phenylethyl)amino-1H-purine

In a 50 ml round-bottomed flask, 500 mg of 2,6-dichloro-1H-purine were dissolved in 10 ml of butanol and 1 ml of propan-2-ol, then 375 μl of 1(R,S)-phenylethylamine were added and the mixture heated to 75° C. After heating for around 3 h, a white precipitate began to appear. After heating for 4 h, the reaction was complete (TLC on 60F254 silica plate—dichloromethane/methanol eluent, 90/10 by volume). After purifying by flash chromatography over silica gel while eluting with a mixture of dichloromethane and methanol (97.5/2.5 by volume), 257 mg of 2-chloro-6-(1(R,S)-phenylethyl)amino-1H-purine, was obtained in the form of a white powder, the characteristics of which were the following:

Melting point (Kofler)=203-204° C.

2-chloro-6-(1(R,S)-phenylethyl)amino-1H-purine may also be obtained according to CA (1971), 74, 31728a (F=199-202° C.).

The products from Examples 4, 5, 6, 7, 8 and 9:

### EXAMPLE 4

2-chloro-6-[2-(morpholin-4-yl)ethylamino]-1H-purine

### EXAMPLE 5

6-(thiophen-2-yl)methylamino-1H-purine

### EXAMPLE 6

2-chloro-6-[2-phenylmethylamino)-ethylamino]-1H-purine

### EXAMPLE 7

6-{2-[3-(3,5-dimethyl-phenyl)oxypropyl]amino}-1H-purine

### EXAMPLE 8

6-[4-(ethyloxycarbonyl)methylpiperidin-1-yl]-1H-purine

### EXAMPLE 9

6-(piperidin-1-yl)-1H-purine
   were attained by proceeding as in Example 3 but by replacing the 1-phenylethylamine with the corresponding starting amines.

Biological Assay for Biologically Characterizing the Invention:

The inorganic phosphate released during the hydrolysis of ATP by the ATPase activity of Hsp82 is quantified by the malachite green method. In the presence of this reagent, formation of the inorganic phosphate-molybdate-malachite green complex occurs, which complex absorbs at a wavelength of 620 nm.

The products to be evaluated are incubated in a reaction volume of 30 μl, in the presence of 1 μM Hsp82 and 250 μM of substrate (ATP) in a buffer composed of 50 mM Hepes-NaOH (pH 7.5), 1 mM DTT, 5 mM MgCl₂ and 50 mM KCl at 37° C. for 60 min. At the same time, a range of inorganic phosphate between 1 and 40 μM is composed in the same buffer. The ATPase activity is then detected by the addition of 60 μl of the Biomel Green reagent (Tebu). After incubating for 20 min at room temperature, the absorbance of the various wells is measured using a microplate reader at 620 nm. The concentration of inorganic phosphate of each sample is then calculated from the standard curve.

The ATPase activity of Hsp82 is expressed as concentration of inorganic phosphate produced in 60 min. The effect of the various products tested is expressed as percentage inhibition of the ATPase activity.

The formation of ADP due to the ATPase activity of Hsp82 was used to develop another method for evaluating the enzymatic activity of this enzyme by application of an enzymatic coupling system involving pyruvate kinase (PK) and lactate dehydrogenase (LDH). In this kinetic-type spectrophotometric method, the PK catalyzes the formation of ATP and of pyruvate from phosphoenol pyruvate (PEP) and from the ADP produced by Hsp82. The pyruvate formed, which is a substrate for LDH, is then converted to lactate in the presence of NADH. In this case, the decrease in NADH concentration, measured by the decrease in absorbance at the wavelength of 340 nm, is proportional to the concentration of ADP produced by Hsp82.

The products tested were incubated in a reaction volume of 100 μl of buffer composed of 100 mM Hepes-NaOH (pH 7.5), 5 mM MgCl₂, 1 mM DTT, 150 mM KCl, 0.3 mM NADH, 2.5 mM PEP and 250 μM ATP. This mixture was preincubated at 37° C. for 30 min before adding 3.77 units of LDH and 3.77 units of PK. The reaction was initiated by addition of the product to be evaluated, in variable concentrations, and of Hsp82, at the concentration of 1 μM. Measurement of the enzymatic activity of Hsp82 was then carried out, continuously, in a microplate reader at 37° C., at the wavelength of 340 nm. The initial rate of the reaction was obtained by measuring the slope of the tangent at the origin of the curve recorded. The enzymatic activity was expressed in μM of ADP formed per minute. The effect of the various products tested was expressed as a percentage inhibition of the ATPase activity.

The inhibitory activities on the ATPase activity of Hsp82 obtained with the products of the invention in the enzymatic coupling system are given in the table below, according to the criteria below for inhibition of the ATPase activity of Hsp82:

| Example | Structure | IC50 μM |
|---|---|---|
| 1 | 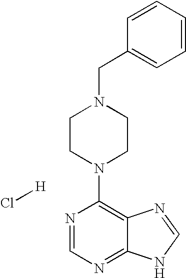 | B |
| 2 | 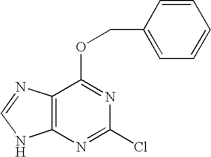 | C |
| 3 | 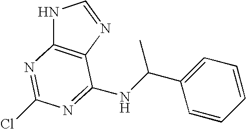 | C |
| 4 | 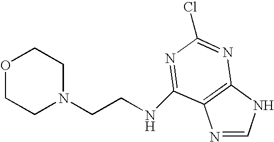 | C |
| 5 | 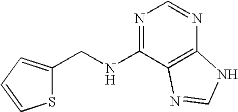 | C |
| 6 | 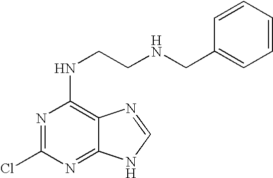 | C |
| 7 | 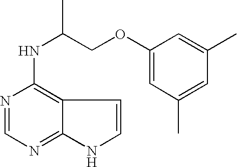 | C |
| 8 | 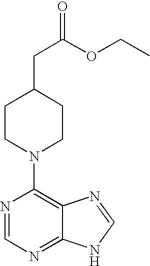 | C |
| 9 | 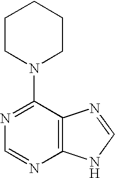 | B |
| 10 | 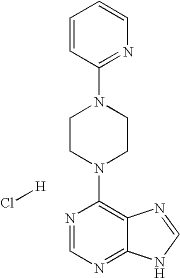 | B |

A: IC50 < 1 μM
B: 1 μM < IC50 < 10 μM
C: 10 μM < IC50 < 100 μM

1. A method of treating a disease, in which an abnormal amount of Hsp90 chaperone protein is involved, in a patient in need thereof, comprising administering to such patient a pharmaceutically effective amount of a compound of formula (IA) 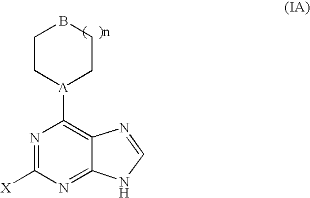
wherein:
X represents a hydrogen atom, a methyl or trifluoromethyl radical;
A represents N or CH;
B represents O, S, NR′, CH₂ or CHR′;
R′ represents a hydrogen atom, or a C₁-C₇ alkyl radical, or a C₂-C₇ alkenyl or alkynyl radical, or a (CH₂)ₙ-aryl or heteroaryl radical, or a C(Z)-aryl or heteroaryl radical; where the aryl or heteroaryl rings, which may be monocyclic or bicyclic with 5 to 10 ring members, may contain from 0 to 3 identical or different heteroatoms chosen from O, S and N, and may optionally be substituted by one or more halogen atoms or by one or more radicals chosen from the group consisting of alkyl, OH, Oalkyl, SH, Salkyl, NH₂, NHalkyl, N(alkyl)₂, CF₃, CN, NO₂, COOH, C(O)Oalkyl, CONH₂, C(O)NHalkyl, C(O)N(alkyl)₂, S(O)alkyl, S(O)₂alkyl, SONH₂, S(O)₂NH₂, S(O)₂NHalkyl, S(O)₂N(alkyl)₂, —C(O)NH₂, P(O)(OH)₂, P(O)(alkyl)OH, P(O)(Oalkyl)₂, P(O)(alkyl)Oalkyl, NH—C(O)—NH₂, NH(CO)NHalkyl, NH(CO)N(alkyl)₂, O—C(O)NHalkyl, and O—C(O)N(alkyl)₂, of which the alkyl parts may be C₁-C₃;
n=0, 1 or 2;
Z represents an oxygen or sulfur atom or an NR′ radical; and
R1 represents a hydrogen atom or a C₁-C₃ alkyl radical; or
a racemate, enantiomer, diastereomer, tautomer or prodrug of such compound, or a pharmaceutically acceptable salt of such compound, racemate, enantiomer, diastereomer, tautomer or prodrug.

2. A method of treating a disease, in which an abnormal amount of Hsp90 chaperone protein is involved, in a patient in need thereof, comprising administering to such patient a pharmaceutically effective amount of a compound of formula (IB) 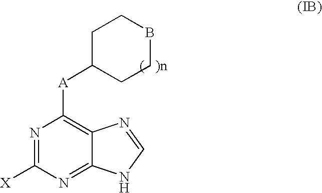
wherein:
X represents a hydrogen atom, a methyl or trifluoromethyl radical;
A represents O, S, NH, CH₂ or CHR;
B represents O, S, NR′, CH₂ or CHR′;
R represents a hydrogen atom, or a C₁-C₃ alkyl radical;
R′ represents a hydrogen atom, or a C₁-C₇ alkyl radical, or a C₂-C₇ alkenyl or alkynyl radical, or a (CH₂)ₙ-aryl or heteroaryl radical, or a C(Z)-aryl or heteroaryl radical; where the aryl or heteroaryl rings, which may be monocyclic or bicyclic with 5 to 10 ring members, may contain from 0 to 3 identical or different heteroatoms chosen from O, S and N, and may optionally be substituted by one or more halogen atoms or by one or more radicals chosen from the group consisting of alkyl, OH, Oalkyl, SH, Salkyl, NH₂, NHalkyl, N(alkyl)₂, CF₃, CN, NO₂, COOH, C(O)Oalkyl, CONH₂, C(O)NHalkyl, C(O)N(alkyl)₂, S(O)alkyl, S(O)₂alkyl, SONH₂, S(O)₂NH₂, S(O)₂NHalkyl, S(O)₂N(alkyl)₂, —C(O)NH₂, P(O)(OH)₂, P(O)(alkyl)OH, P(O)(Oalkyl)₂, P(O)(alkyl)Oalkyl, NH—C(O)—NH₂, NH(CO)NHalkyl, NH(CO)N(alkyl)₂, O—C(O)NHalkyl, and O—C(O)N(alkyl)₂, of which the alkyl parts may be C₁-C₃;
n=0, 1 or 2; and
Z represents an oxygen or sulfur atom or an NR′ radical; or
a racemate, enantiomer, diastereomer, tautomer or prodrug of such compound, or a pharmaceutically acceptable salt of such compound, racemate, enantiomer, diastereomer, tautomer or prodrug.

3. A method of treating a disease, in which an abnormal amount of Hsp90 chaperone protein is involved, in a patient in need thereof, comprising administering to such patient a pharmaceutically effective amount of a compound of formula (II) 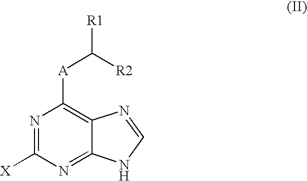
wherein:
X represents a hydrogen or halogen atom, a methyl or trifluoromethyl radical;
A represents O, S, NH, NR1, CH₂ or CHR1;
n=0, 1 or 2;
R1 represents a hydrogen atom or a C₁-C₃ alkyl radical; and
R2 represents a C₁-C₃ alkyl radical or a CHR1-aryl or heteroaryl ring; where the aryl or heteroaryl ring, which may be monocyclic or bicyclic with 5 to 10 ring members, may contain from 0 to 3 identical or different heteroatoms chosen from O, S and N; the alkyl radical or the aryl or heteroaryl ring may optionally be substituted by one or more halogen atoms or by one or more radicals chosen from alkyl, OH, Oalkyl, SH, Salkyl, NH₂, NHalkyl, N(alkyl)₂, CF₃, CN, NO₂, COOH, C(O)Oalkyl, CONH₂, C(O)NHalkyl, C(O)N(alkyl)₂, S(O)alkyl, S(O)₂alkyl, SONH₂, S(O)₂NH₂, S(O)₂NHalkyl, S(O)₂N(alkyl)₂, —C(O)NH₂, P(O)(OH)₂, P(O)(alkyl)OH, P(O)(Oalkyl)₂, P(O)(alkyl)Oalkyl, NH—C(O)—NH₂, NH(CO)NHalkyl, NH(CO)N(alkyl)₂, O—C(O)NHalkyl, and O—C(O)N(alkyl)₂, of which the alkyl parts may be C₁-C₃; or
a racemate, enantiomer, diastereomer, tautomer or prodrug of such compound, or a pharmaceutically acceptable salt of such compound, racemate, enantiomer, diastereomer, tautomer or prodrug.

4. A method according to claim 1 wherein X is H.

5. A method according to claim 2 wherein X is H.

6. A method according to claim 3 wherein X is Cl.

7. A method according to claim 1 wherein the compound is chosen from
6-[4-(ethyloxycarbonyl)methylpiperidin-1-yl]1H-purine;
6-(piperidin-1-yl)-1H-purine; and
is 6-[4-(pyridin-2-yl)piperazinyl]-1-H-purine monohydrochloride; or
a tautomer or prodrug of such compound, or a pharmaceutically acceptable salt of such compound, tautomer or prodrug.

8. A method according to claim 3 wherein the compound is chosen from
6-(phenylmethyl)amino-1H-purine;
2-chloro-6-phenylmethyloxy-1H-purine;
2-chloro-6-(1(R,S)-phenylethyl)amino-1H-purine;
2-chloro-6-[2-(morpholin-4-yl)ethylamino]-1H-purine;
6-(thiophen-2-yl)methylamino-1H-purine;
2-chloro-6-[2-(phenylmethylamino)ethylamino]-1H-purine; and
6-{2-[3-(3,5-dimethylphenyl)oxypropyl]amino}-1H-purine; or
a racemate, enantiomer, diastereomer, tautomer or prodrug of such compound, or a pharmaceutically acceptable salt of such compound, racemate, enantiomer, diastereomer, tautomer or prodrug.

9. A method for inhibiting the Hsp90 chaperone, in a patient in need of such inhibition, comprising administering to said patient a pharmaceutically effective amount of a compound of formula (IA) 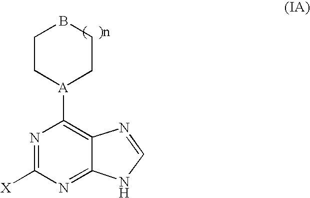
wherein:
X represents a hydrogen atom, a methyl or trifluoromethyl radical;
A represents N or CH;
B represents O, S, NR′, CH₂ or CHR′;
R′ represents a hydrogen atom, or a C₁-C₇ alkyl radical, or a C₂-C₇ alkenyl or alkynyl radical, or a (CH₂)ₙ-aryl or heteroaryl radical, or a C(Z)-aryl or heteroaryl radical; where the aryl or heteroaryl rings, which may be monocyclic or bicyclic with 5 to 10 ring members, may contain from 0 to 3 identical or different heteroatoms chosen from O, S and N, and may optionally be substituted by one or more halogen atoms or by one or more radicals chosen from the group consisting of alkyl, OH, Oalkyl, SH, Salkyl, NH₂, NHalkyl, N(alkyl)₂, CF₃, CN, NO₂, COOH, C(O)Oalkyl, CONH₂, C(O)NHalkyl, C(O)N(alkyl)₂, S(O)alkyl, S(O)₂alkyl, SONH₂, S(O)₂NH₂, S(O)₂NHalkyl, S(O)₂N(alkyl)₂, —C(O)NH₂, P(O)(OH)₂, P(O)(alkyl)OH, P(O)(Oalkyl)₂, P(O)(alkyl)Oalkyl, NH—C(O)—NH₂, NH(CO)NHalkyl, NH(CO)N(alkyl)₂, O—C(O)NHalkyl, and O—C(O)N(alkyl)₂, of which the alkyl parts may be C₁-C₃;
n=0, 1 or 2;
Z represents an oxygen or sulfur atom or an NR′ radical; and
R1 represents a hydrogen atom or a C₁-C₃ alkyl radical; or
a racemate, enantiomer, diastereomer, tautomer or prodrug of such compound, or a pharmaceutically acceptable salt of such compound, racemate, enantiomer, diastereomer, tautomer or prodrug.

10. A method for inhibiting the Hsp90 chaperone, in a patient in need of such inhibition, comprising administering to said patient a pharmaceutically effective amount of a compound of formula (IB) 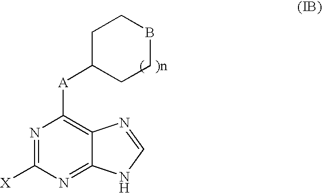
wherein:
X represents a hydrogen atom, a methyl or trifluoromethyl radical;
A represents O, S, NH, CH₂ or CHR;
B represents O, S, NR′, CH₂ or CHR′;
R represents a hydrogen atom, or a C₁-C₃ alkyl radical;
R′ represents a hydrogen atom, or a C₁-C₇ alkyl radical, or a C₂-C₇ alkenyl or alkynyl radical, or a (CH₂)ₙ-aryl or heteroaryl radical, or a C(Z)-aryl or heteroaryl radical; where the aryl or heteroaryl rings, which may be monocyclic or bicyclic with 5 to 10 ring members, may contain from 0 to 3 identical or different heteroatoms chosen from O, S and N, and may optionally be substituted by one or more halogen atoms or by one or more radicals chosen from the group consisting of alkyl, OH, Oalkyl, SH, Salkyl, NH₂, NHalkyl, N(alkyl)₂, CF₃, CN, NO₂, COOH, C(O)Oalkyl, CONH₂, C(O)NHalkyl, C(O)N(alkyl)₂, S(O)alkyl, S(O)₂alkyl, SONH₂, S(O)₂NH₂, S(O)₂NHalkyl, S(O)₂N(alkyl)₂, —C(O)NH₂, P(O)(OH)₂, P(O)(alkyl)OH, P(O)(Oalkyl)₂, P(O)(alkyl)Oalkyl, NH—C(O)—NH₂. NH(CO)NHalkyl, NH(CO)N(alkyl)₂, O—C(O)NHalkyl, and O—C(O)N(alkyl)₂, of which the alkyl parts may be C₁-C₃;
n=0, 1 or 2; and
Z represents an oxygen or sulfur atom or an NR′ radical; or
a racemate, enantiomer, diastereomer, tautomer or prodrug of such compound, or a pharmaceutically acceptable salt of such compound, racemate, enantiomer, diastereomer, tautomer or prodrug.

11. A method for inhibiting the Hsp90 chaperone, in a patient in need of such inhibition, comprising administering to said patient a pharmaceutically effective amount of a compound of formula (II) 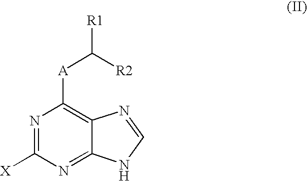
wherein:
X represents a hydrogen or halogen atom, a methyl or trifluoromethyl radical;
A represents O, S, NH, NR1, CH₂ or CHR1;
n=0, 1 or 2;
R1 represents a hydrogen atom or a C₁-C₃ alkyl radical; and
R2 represents a C₁-C₃ alkyl radical or a CHR1-aryl or heteroaryl ring; where the aryl or heteroaryl ring, which may be monocyclic or bicyclic with 5 to 10 ring members, may contain from 0 to 3 identical or different heteroatoms chosen from O, S and N; the alkyl radical or the aryl or heteroaryl ring may optionally be substituted by one or more halogen atoms or by one or more radicals chosen from alkyl, OH, Oalkyl, SH, Salkyl, NH₂, NHalkyl, N(alkyl)₂, CF₃, CN, NO₂, COOH, C(O)Oalkyl, CONH₂, C(O)NHalkyl, C(O)N(alkyl)₂, S(O)alkyl, S(O)₂alkyl, SONH₂, S(O)₂NH₂, S(O)₂NHalkyl, S(O)₂N(alkyl)₂, —C(O)NH₂, P(O)(OH)₂, P(O)(alkyl)OH, P(O)(Oalkyl)₂, P(O)(alkyl)Oalkyl, NH—C(O)—NH₂, NH(CO)NHalkyl, NH(CO)N(alkyl)₂, O—C(O)NHalkyl, and O—C(O)N(alkyl)₂, of which the alkyl parts may be C₁-C₃; or
a racemate, enantiomer, diastereomer, tautomer or prodrug of such compound, or a pharmaceutically acceptable salt of such compound, racemate, enantiomer, diastereomer, tautomer or prodrug.

12. A method of treating cancer, in a patient in need of such treatment, comprising administering to such patient a pharmaceutically effective amount of a compound of formula (IA) 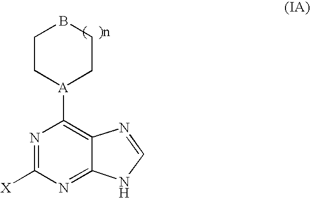
wherein:
X represents a hydrogen atom, a methyl or trifluoromethyl radical;
A represents N or CH;
B represents O, S, NR′, CH₂ or CHR′;
R′ represents a hydrogen atom, or a C₁-C₇ alkyl radical, or a C₂-C₇ alkenyl or alkynyl radical, or a (CH₂)ₙ-aryl or heteroaryl radical, or a C(Z)-aryl or heteroaryl radical; where the aryl or heteroaryl rings, which may be monocyclic or bicyclic with 5 to 10 ring members, may contain from 0 to 3 identical or different heteroatoms chosen from O, S and N, and may optionally be substituted by one or more halogen atoms or by one or more radicals chosen from the group consisting of alkyl, OH, Oalkyl, SH, Salkyl, NH₂, NHalkyl, N(alkyl)₂, CF₃, CN, NO₂, COOH, C(O)Oalkyl, CONH₂, C(O)NHalkyl, C(O)N(alkyl)₂, S(O)alkyl, S(O)₂alkyl, SONH₂, S(O)₂NH₂, S(O)₂NHalkyl, S(O)₂N(alkyl)₂, —C(O)NH₂, P(O)(OH)₂, P(O)(alkyl)OH, P(O)(Oalkyl)₂, P(O)(alkyl)Oalkyl, NH—C(O)—NH₂, NH(CO)NHalkyl, NH(CO)N(alkyl)₂, O—C(O)NHalkyl, and O—C(O)N(alkyl)₂, of which the alkyl parts may be C₁-C₃;
n=0, 1 or 2;
Z represents an oxygen or sulfur atom or an NR′ radical; and
R1 represents a hydrogen atom or a C₁-C₃ alkyl radical; or
a racemate, enantiomer, diastereomer, tautomer or prodrug of such compound, or a pharmaceutically acceptable salt of such compound, racemate, enantiomer, diastereomer, tautomer or prodrug.

13. A method of treating cancer, in a patient in need of such treatment, comprising administering to such patient a pharmaceutically effective amount of a compound of formula (IB) 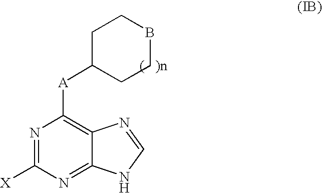
wherein:
X represents a hydrogen atom, a methyl or trifluoromethyl radical;
A represents O, S, NH, CH₂ or CHR;
B represents O, S, NR′, CH₂ or CHR′;
R represents a hydrogen atom, or a C₁-C₃ alkyl radical;
R′ represents a hydrogen atom, or a C₁-C₇ alkyl radical, or a C₂-C₇ alkenyl or alkynyl radical, or a (CH₂)ₙ-aryl or heteroaryl radical, or a C(Z)-aryl or heteroaryl radical; where the aryl or heteroaryl rings, which may be monocyclic or bicyclic with 5 to 10 ring members, may contain from 0 to 3 identical or different heteroatoms chosen from O, S and N, and may optionally be substituted by one or more halogen atoms or by one or more radicals chosen from the group consisting of alkyl, OH, Oalkyl, SH, Salkyl, NH₂, NHalkyl, N(alkyl)₂, CF₃, CN, NO₂, COOH, C(O)Oalkyl, CONH₂, C(O)NHalkyl, C(O)N(alkyl)₂, S(O)alkyl, S(O)₂alkyl, SONH₂, S(O)₂NH₂, S(O)₂NHalkyl, S(O)₂N(alkyl)₂, —C(O)NH₂, P(O)(OH)₂, P(O)(alkyl)OH, P(O)(Oalkyl)₂, P(O)(alkyl)Oalkyl, NH—C(O)—NH₂, NH(CO)NHalkyl, NH(CO)N(alkyl)₂, O—C(O)NHalkyl, and O—C(O)N(alkyl)₂, of which the alkyl parts may be C₁-C₃;
n=0, 1 or 2; and
Z represents an oxygen or sulfur atom or an NR′ radical; or
a racemate, enantiomer, diastereomer, tautomer or prodrug of such compound, or a pharmaceutically acceptable salt of such compound, racemate, enantiomer, diastereomer, tautomer or prodrug.

14. A method of treating cancer, in a patient in need of such treatment, comprising administering to such patient a pharmaceutically effective amount of a compound of formula (II) 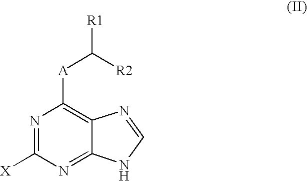
wherein:
X represents a hydrogen or halogen atom, a methyl or trifluoromethyl radical;
A represents O, S, NH, NR1, CH₂ or CHR1;
n=0, 1 or 2;
R1 represents a hydrogen atom or a C₁-C₃ alkyl radical; and
R2 represents a C₁-C₃ alkyl radical or a CHR1-aryl or heteroaryl ring; where the aryl or heteroaryl ring, which may be monocyclic or bicyclic with 5 to 10 ring members, may contain from 0 to 3 identical or different heteroatoms chosen from O, S and N; the alkyl radical or the aryl or heteroaryl ring may optionally be substituted by one or more halogen atoms or by one or more radicals chosen from alkyl, OH, Oalkyl, SH, Salkyl, NH₂, NHalkyl, N(alkyl)₂, CF₃, CN, NO₂, COOH, C(O)Oalkyl, CONH₂, C(O)NHalkyl, C(O)N(alkyl)₂, S(O)alkyl, S(O)₂alkyl, SONH₂, S(O)₂NH₂, S(O)₂NHalkyl, S(O)₂N(alkyl)₂, —C(O)NH₂, P(O)(OH)₂, P(O)(alkyl)OH, P(O)(Oalkyl)₂, P(O)(alkyl)Oalkyl, NH—C(O)—NH₂, NH(CO)NHalkyl, NH(CO)N(alkyl)₂, O—C(O)NHalkyl, and O—C(O)N(alkyl)₂, of which the alkyl parts may be C₁-C₃; or
a racemate, enantiomer, diastereomer, tautomer or prodrug of such compound, or a pharmaceutically acceptable salt of such compound, racemate, enantiomer, diastereomer, tautomer or prodrug.

15. A method of treating cancer, in a patient in need of such treatment, comprising administering to such patient a pharmaceutically effective amount of a compound chosen from
6-[4-(ethyloxycarbonyl)methylpiperidin-1-yl]1H-purine;
6-(piperidin-1-yl)-1H-purine; and
6-[4-(pyridin-2-yl)piperazinyl]-1-H-purine monohydrochloride; or
a tautomer or prodrug of such compound, or a pharmaceutically acceptable salt of such compound, tautomer or prodrug.

16. A method of treating cancer, in a patient in need of such treatment, comprising administering to such patient a pharmaceutically effective amount of a compound chosen from
6-(phenylmethyl)amino-1H-purine;
2-chloro-6-phenylmethyloxy-1H-purine;
2-chloro-6-(1(R,S)-phenylethyl)amino-1H-purine;
2-chloro-6-[2-(morpholin-4-yl)ethylamino]-1H-purine;
6-(thiophen-2-yl)methylamino-1H-purine;
2-chloro-6-[2-(phenylmethylamino)ethylamino]-1H-purine; and
6-{2-[3-(3,5-dimethylphenyl)oxypropyl]amino}-1H-purine; or
a racemate, enantiomer, diastereomer, tautomer or prodrug of such compound, or a pharmaceutically acceptable salt of such compound, racemate, enantiomer, diastereomer, tautomer or prodrug.
